(19) United States
(12) Patent Application Publication
(10) Pub. No.: US 2024/0172950 A1
(43) Pub. Date: May 30, 2024

(54) SYSTEMS AND METHODS FOR VEHICLE OCCUPANT VITAL SIGN DETECTION

(71) Applicant: VEONEER US, LLC, Southfield, MI (US)

(72) Inventor: Shan Cong, Superior Township, MI (US)

(21) Appl. No.: 18/072,662

(22) Filed: Nov. 30, 2022

(51) Int. Cl. A61B 5/05; A61B 5/00; A61B 5/0205; A61B 5/11; G01S 7/41

(52) U.S. Cl. CPC A61B 5/05; A61B 5/0205; A61B 5/1113; A61B 5/6893; A61B 5/7264; G01S 7/415; A61B 5/024

(57) ABSTRACT

Methods and systems for detection and/or estimation of a rate of an occupant vital sign from within a cabin of a vehicle. In some implementations, the method may comprise identifying a repeating pattern of Doppler spectrum peaks in RADAR signal using a plurality of different range bins. An estimated frequency distance between adjacent peaks of the repeating pattern may then be identified using the signal data. An estimated rate of a repeating vital sign of an occupant within the cabin of the vehicle may then be calculated using the estimated frequency distance.

### SUMMARY

RADAR is often used to detect objects exterior to a vehicle, such as other vehicles, pedestrians, and obstacles. However, RADAR, or other electromagnetic radiation signals, are not typically directed inward toward occupants of the cabin, let alone to monitor important conditions that may impact the safety of the occupants, such as breathing rates, heart rates, or other vital signs.

The present inventors have therefore determined that it would be desirable to provide systems and methods that overcome one or more of the foregoing limitations and/or other limitations of the prior art. Systems and methods for detecting and/or monitoring a vital sign of an occupant within a vehicle are therefore disclosed herein.

Thus, in some embodiments, the inventive concepts disclosed herein may be used to detect vital signs of one or more occupants within a vehicle, estimate a vital sign rate of such vital sign(s), classify vehicle occupants using, at least in part, such data, and/or take various actions using such data.

In a more particular example of a method for detection of an occupant vital sign from within a cabin of a vehicle using RADAR, the method may comprise identifying a repeating pattern of Doppler spectrum peaks in RADAR signal over a plurality of different range bins. An estimated frequency distance between adjacent peaks of the repeating pattern, such as a repeating pattern within the same range bin in some cases, may then be identified. An estimated rate of a repeating vital sign of one or more occupants within the cabin of the vehicle may then be calculated using the estimated frequency distance.

In some implementations, the estimated repeating vital sign may comprise a breathing rate. Alternatively, the estimated repeating vital sign may comprise, for example, a heart rate.

In some implementations, the step of identifying a repeating pattern of Doppler spectrum peaks may comprise selecting a strongest repeating signal from among a plurality of RADAR signals. Some implementations may further comprise selecting one or more other signals sufficiently related to the strongest repeating signal. For example, some implementations may comprise selecting at least one range bin adjacent to a range bin associated with the strongest signal and identifying an estimated frequency distance between adjacent peaks of a repeating pattern from a RADAR signal in the at least one range bin adjacent to the range bin associated with the strongest signal.

Some implementations may further comprise using the estimated frequency distance between adjacent peaks of a repeating RADAR signal in the at least one range bin adjacent to the range bin associated with the strongest signal to improve accuracy of at least one estimated parameter derived from the repeating RADAR signal.

In some implementations, the at least one estimated parameter may comprise a location of the occupant within the vehicle.

Some implementations may further comprise classifying the occupant using the estimated repeating vital sign.

In an example of a method for detection of an occupant vital sign from within a cabin of a vehicle, the method may comprise transmitting one or more electromagnetic signals within a cabin of a vehicle and processing signals associated with the one or more electromagnetic signals, such as signals reflected from the one or more electromagnetic signals. A distance between adjacent signal frequency peaks may be derived from the reflected signals, which may be indicative of a vital sign of an occupant within the cabin of the vehicle. A rate of the vital sign of the occupant may then be estimated using the distance between adjacent frequency peaks.

In some implementations in which reflected signals are received, the step of processing reflected signals may comprise processing reflected signals from the one or more electromagnetic signals in a plurality of range bins. As described throughout this disclosure, this may be done, in some embodiments, by identifying an estimated frequency distance between adjacent Doppler peaks. In some such implementations, the step of processing reflected signals may comprise processing reflected signals from the one or more electromagnetic signals in a first range bin corresponding with a target range bin and a second range bin adjacent to the target range bin. Some implementations may further comprise identifying the target range bin by comparing signal strengths of the reflected signals.

In some implementations, each of the one or more electromagnetic signals may comprise RADAR signals.

Some implementations may further comprise transmitting one or more electromagnetic signals within a cabin of a vehicle to an intended direction corresponding with an anticipated location of an occupant. For example, after detecting a signal indicative of a possible vital sign, the RADAR or other electromagnetic sensor may be tuned to the location of the occupant associated with this signal.

In some implementations, the rate of the vital sign may comprise a breathing rate. Alternatively, the rate of the vital sign may comprise a heart rate.

In an example of a system for in-cabin detection of occupant vital signs using electromagnetic signal processing, the system may comprise one or more electromagnetic sensors, such as RADAR modules, positioned within a cabin of a vehicle. The system may further comprise a detection module configured to process reflected electromagnetic signals into a plurality of range bins and/or a vital sign module configured to use a signal repetition frequency, such as a distance between adjacent frequency peaks, associated with one or more of the range bins and estimate a rate associated with a vital sign of an occupant within the cabin of the vehicle.

Some embodiments may further comprise a classification module, which may be configured to receive information from the vital sign module and classify the occupant according to an age group using the rate associated with the vital sign of the occupant.

The vital sign module may be configured to estimate a breathing rate and/or a heart rate of the occupant.

In some embodiments, the electromagnetic sensor may be configured to tune a signal to a target location expected to correspond with a location of the occupant.

The features, structures, steps, or characteristics disclosed herein in connection with one embodiment may be combined in any suitable manner in one or more alternative embodiments.

### BRIEF DESCRIPTION OF THE DRAWINGS

Non-limiting and non-exhaustive embodiments of the disclosure are described, including various embodiments of the disclosure with reference to the figures, in which:

FIG. 1 depicts a vehicle comprising a system for detection of vehicle occupant vital signs according to some embodiments;

FIG. 2 is a graph representing Doppler spectrum signals received from a vehicle occupant;

FIG. 3 is a graph representing a series of signals, each in a different range bin, representing data from which a vehicle occupant vital sign may be derived;

FIG. 4 is a chart illustrating a statistical correlation between age and breathing rates in breaths per minute that may be used to classify vehicle occupants according to a vital sign rate;

FIG. 5 is a block diagram illustrating an example of a system for estimating a vital sign of an occupant within a vehicle according to some embodiments;

FIG. 6 depicts an example of a vehicle comprising a system for estimating a vital sign rate of an occupant in a vehicle; and

FIG. 7 is a flow chart depicting an example of a method for using electromagnetic radiation to detect and estimate a vital sign of a vehicle occupant according to some implementations.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

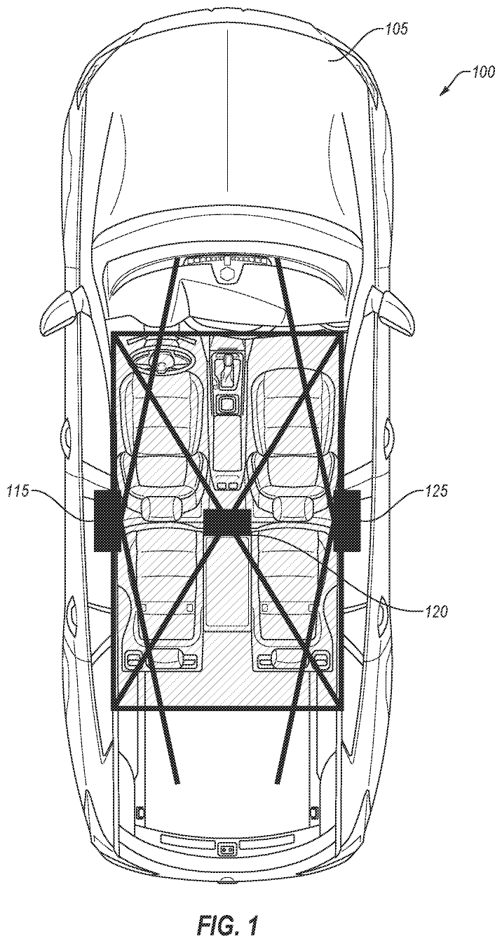

FIG. 1 depicts a system 100 for detection of occupant vital signs within the cabin of a vehicle 105. As shown in this figure, one or more sensors may be positioned at various locations within the cabin of vehicle 105. In the depicted embodiment, vehicle 105 comprises three sensors 115, 120, and 125. Sensor 115 is positioned on one side of the vehicle 105, sensor 120 is positioned at a central location within vehicle 105, such as mounted on the ceiling of the cabin, and sensor 125 is mounted on the opposite side of vehicle 105. As those of ordinary skill in the art will appreciate, however, a wide variety of alternatives are possible, including different numbers of sensors, different types of sensors, and different locations of sensors.

For example, in preferred embodiments, sensors 115, 120, and 125 may comprise RADAR sensors, such as a frequency modulated continuous wave (FMCW) ultra-wide band RADAR sensor configured to operate at 60 GHz. However, in alternative embodiments, other types of sensors may be used, such as LIDAR or other types of electromagnetic sensors, for example. In addition, in some embodiments, a single sensor may be used, such as sensor 120, or two sensors (one in the middle and one on just one side of the vehicle, for example). More than three sensors may also be used in some embodiments. Although it may be preferably to locate the sensors in the roof/ceiling or the upper side of a vehicle pillar, in some such embodiments, or in alternative embodiments having three or fewer sensors, such sensor(s) may alternatively be positioned in the front of the vehicle, the rear of the vehicle, within seats of the vehicle, and/or in the floor of the vehicle, for example.

As illustrated by the various lines extending from sensors 115/120/125, each of these sensors may be configured to direct electromagnetic signals to and/or receive electromagnetic signals from particular regions of the cabin of vehicle 105, preferably so as to at least be capable of detecting occupants within each of the seats of the vehicle 105. Of course, again, many alternatives are contemplated and/or would be available to those of ordinary skill in the art after having received the benefit of this disclosure. For example, a single sensor positioned at a suitable location may, for some vehicles, be sufficient to adequately detect occupants in every seat in the vehicle. Similarly, in other embodiments, it may be desirable to provide a dedicated RADAR or other electromagnetic sensor for each seat of the vehicle.

As described in greater detail below, irrespective of the placement, number, and type of electromagnetic sensors used in the vehicle, in preferred embodiments, such sensor(s) may be used to identify one or more occupants present in the cabin and identify vital sign data about such occupant(s). This vital sign data, such as breathing rates, tidal volume changes, and/or heart rates, for example, may be used to classify the occupant(s). For example, vehicle occupants may be classified according to their age group. By doing so, various actions may be taken and/or vehicle functions changed based upon the classification and/or location of the vehicle occupant(s).

Vital sign data may be collected, for example, using RADAR frequency response data from vital signals of the occupant(s). To provide an example of a model for tracking vital sign signals, consider the following equations:

s(t)=A(t)e^{jϕ(t)}; A(t) is the target amplitude

$$ϕ ( t ) = 2 p \frac{{r}_{0} + r ( t )}{λ} + d ( t ) ;$$

r₀ is the target nominal range.

r(t)=b sin ω0t; r(t) is the target motion over time and b is the maximum motion of the target

δ(t): phase noise

The frequency domain representation of the vital sign signal can then be represented as follows:

$$f ( ω ) = aT {\sum }_{n = - ∞}^{∞} {J}_{- n} ( β ) sin c ( ω - n {ω}_{0} ) \frac{T}{2}$$

where J₋ₙ is Bessel's first integral and

$$β = - \frac{2 π b}{λ}$$

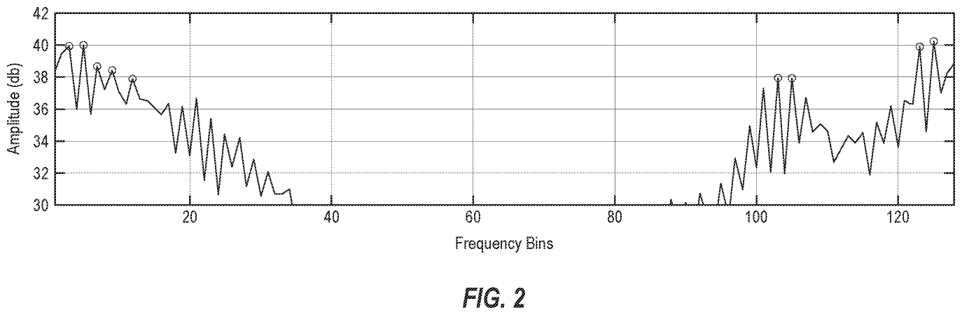

FIG. 2 is a graph representing Doppler spectrum signals received from an adult occupant of a vehicle. In the example provided in the figure, the adult is estimated to have a breathing rate of about 10.15 breaths per minute. As shown in this graph, using the equations above, the breathing rate can be derived from the distance between peaks in the signal or “repetition frequency,” several of which are circled in the graph.

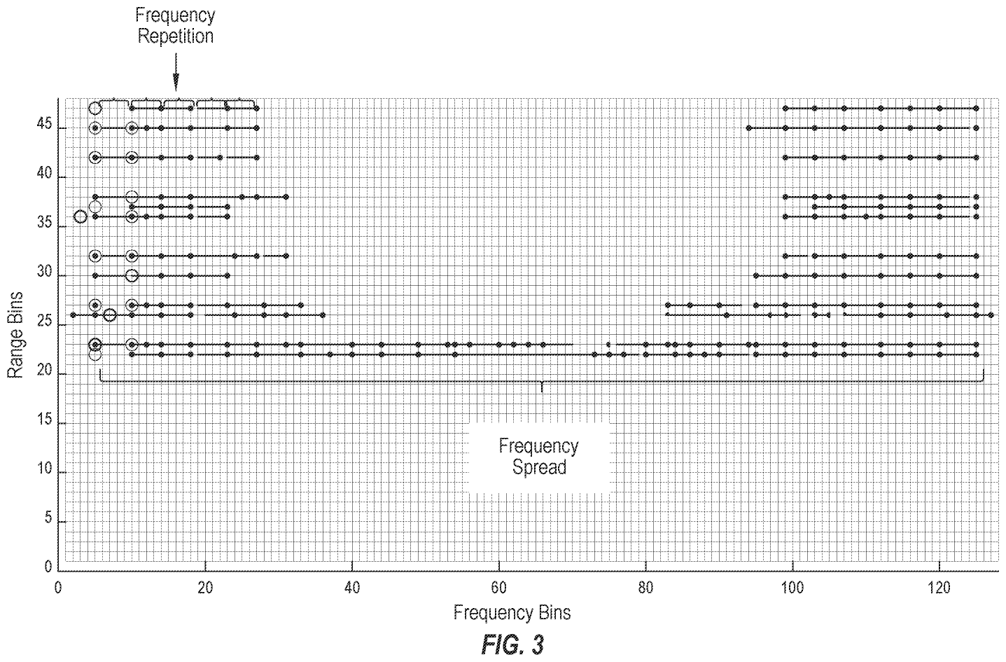

FIG. 3 is another graph representing a series of signals, each in a different range bin. Each range bin represents a distance from the sensor of about 0.047 m and each frequency bin represents a frequency band of about 0.078 Hz. The signal shown on the graph of FIG. 2 is represented at range bin 22 in the graph of FIG. 3, which is the strongest signal detected among those shown in FIG. 3.

The Doppler spectrum peaks are represented by dots in the graph of FIG. 3 and, once again, some of these peaks are circled. This represents Doppler spectrum peaks that passed the detection threshold. In this example, only two Doppler peaks have been allowed per range bin. The frequency repetition, which is represented by the brackets adjacent to range bin 47, can again be derived from the frequency spread shown in FIG. 3. Note that the signals from other range bins generally have the same frequency repetition rate, which may be attributed to other regions of the same occupant's body or from multipath propagation. Of course, some signals may be distinguished as coming from a different occupant or other object within the vehicle. As those of ordinary skill in the art will appreciate, such signals may be processed and/or extracted to be identified as such by way of processing and/or filtering steps disclosed herein or otherwise available to those of ordinary skill in the art.

As mentioned above, although the data shown in FIGS. 2 and 3 represent breathing rates, other vital signs may be estimated using similar techniques. For example, estimated heart rates and/or tidal volume changes may alternatively be derived from RADAR or other electromagnetic signals using the methods disclosed herein.

Once a breathing rate or other vital sign has been estimated for one or more occupants with a vehicle using RADAR or other electromagnetic signal processing, this vital sign data may be used to classify the occupant(s). For example, because respiratory rates are strongly correlated with age, an estimate of the age of the occupant may be made using the estimated breathing rate derived from the RADAR signal processing.

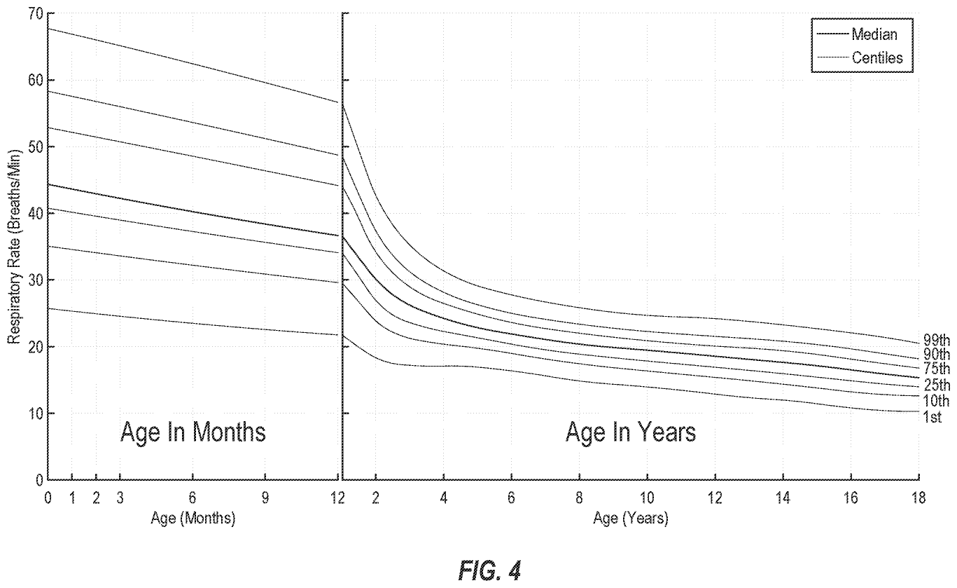

FIG. 4 is a chart illustrating the statistical correlation between age and breathing rates in breaths per minute. This chart illustrates how, for example, a breathing rate of about 40 breaths per minute might reasonably be used to classify an occupant as an infant. Thus, using statistical data, such as from the chart of FIG. 4 or other similar data, a vehicle may be configured to use breathing rates, or other vital sign rates, to classify an occupant according to the occupant's predicted age group. As described in greater detail below, this classification may then be used in various ways by the vehicle to perform useful functions.

For example, if an occupant is identified as being an infant, an airbag may be automatically disabled in the seat associated with this occupant. As another example, if an occupant is identified as an infant or child and remains in the vehicle after the vehicle has been turned off and/or the driver of the vehicle has exited the cabin, the vehicle may be configured to send notifications or warnings regarding the occupant left behind. In some embodiments, the vehicle may be configured to continue to monitor the occupant(s) remaining in the vehicle and only provide such notifications in the event of a detected confluence of data, which may include data from other sensors.

For example, if the vital sign data from the RADAR or other electromagnetic sensor has classified the occupant as an infant or child, rather than immediately sending a notification, the vehicle may be configured to monitor other data, such as temperature and/or time since the child has been left within the vehicle. Upon detecting a temperature within the cabin beyond a threshold temperature, such as 90 degrees Fahrenheit, for example, in combination with data indicative of a child being left in the vehicle, the vehicle may be configured to send a notification to a user. This warning may, for example, be sent to a smart phone of an owner/user of the vehicle. Alternatively, the vehicle may be configured to automatically start the engine and/or start an air conditioning unit of the vehicle in response to a detection of these triggers/thresholds. It should be understood that any of sensors 115/120/125 may therefore comprise a temperature sensor in some embodiments, which may operate in conjunction with RADAR or other electromagnetic sensors to achieve this result.

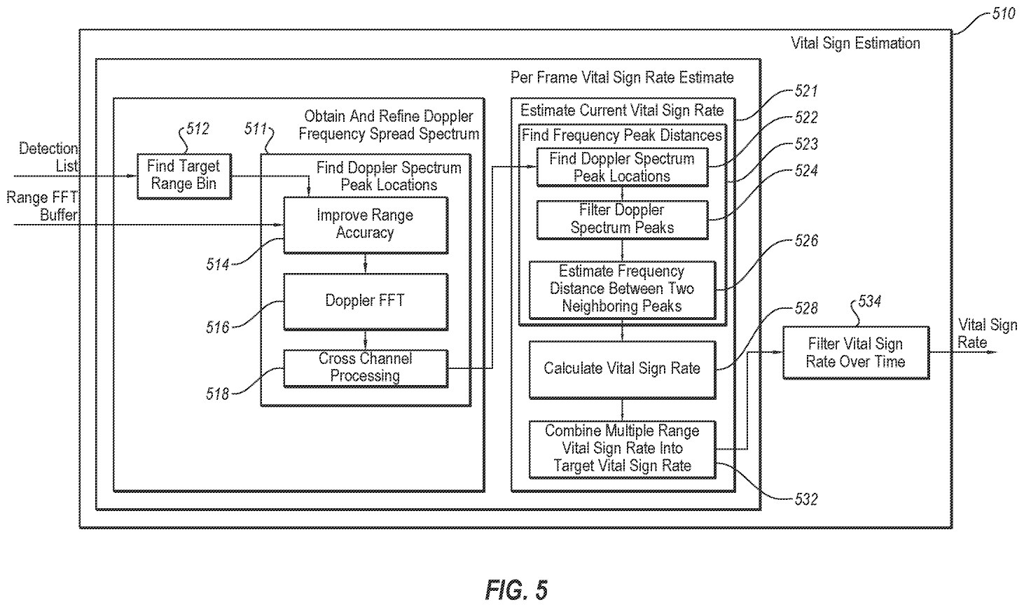

FIG. 5 is a block diagram illustrating an example of a system 510 for estimating a vital sign of an occupant within a vehicle according to some embodiments. As shown in this figure, a detection list comprising a one or more detected signals from a RADAR or other electromagnetic sensor may be fed into a bin, such as a range bin.

At 512, FIG. 5 indicates that the system may be configured to identify a target signal, such as a target range bin or other data set associated with the target and/or target range, from the detection list. In some embodiments and implementations of related methods, this may be done by identifying the strongest signal and/or range bin signal from a list of detections available in the detection structure and having a repeating pattern indicative of a vital sign. Thus, referring back to the chart of FIG. 3, which may be considered an example of a “detection list” for purposes of FIG. 5, the detections in range bin 22 may be identified as the target signal or target bin at 512.

This target signal data, including data gathered and/or processed prior to identification of the target signal/target bin and/or data gathered and/or processed following such identification, may then be used to obtain and refine a Doppler frequency spread spectrum. Thus, for example, the target signal data may be used to identify Doppler spectrum peak locations, as indicated at 511.

In some embodiments and implementations, a fast Fourier transform (FFT) methodology may be used to process the data and output an estimated vital sign. Thus, a buffered range of FFT data may be provided, as also indicated in FIG. 5. In some such embodiments and implementations, the buffered range FFT data may be of the past N frames—for example, N may be equal to 128 frames). In some cases, this data may be associated with a particular target location corresponding with the vehicle occupant the vital sign of which is being monitored. Thus, multiple sets of data may be used, each corresponding with a different occupant. Alternatively, of course, the system 510 may be configured to simply detect the presence of a vehicle occupant without regard to the occupant's location.

In some embodiments and implementations, system 510 may be configured to improve the accuracy of the range at 514. This may comprise, for example, using interpolation techniques or other similar techniques available to those of ordinary skill in the art, such as interpolation techniques that use Quinn's first and second estimators, for example.

In some embodiments and implementations, Doppler FFT may be performed, as indicated at 516 in FIG. 5. This may be performed for the target range bin/data set and, in some preferred embodiments, may also be performed for one or more range bins/data sets adjacent to the target range bin/data set or otherwise sufficiently related to the target range bin/data set to improve the vital sign estimation. For example, in some embodiments, each target range bin/data set within a particular number of bins of the target range bin may be used. Alternatively, each data set within a threshold signal strength of the target range bin/data may be used.

In some embodiments and implementations, cross-channel processing may be performed at 518, preferably over all received signal channels associated with the target range bin/data set and/or one or more adjacent or otherwise sufficiently related to the target range bin/data set. This may be accomplished, for example, by averaging and/or performing cross-channel processing over all received channel signals. Alternatively, this may be accomplished by beamforming the signals to an intended direction, which may be determined following location of the occupant within the vehicle and/or initial signal processing to determine a more precise location associated with a particular occupant to improve signal strength, such as a particular part of the occupant's chest for breathing rate estimation or the occupant's heart for heart rate estimation.

Data from cross-channel processing may then be used to estimate the current vital sign, such as the current breathing rate, at 521. In some embodiments and implementations, this may be achieved by determining frequency peak distances in a set of signal data associated with the target range bin(s)/target data set, as indicated at 523. Thus, for example, Doppler spectrum peaks in the target range bin(s) of interest and/or target signal data set may be identified and/or stored at 522. The Doppler spectrum peaks may then be filtered at 524, which may allow for estimation of the frequency distance, or average frequency distance, between adjacent peaks in the spectrum, or at least a portion of the spectrum/data set, as indicated at 526.

The frequency distance between adjacent peaks in the data set may then be used to calculate an estimated breathing rate or another vital sign for all of the range bins or other data collections of interest, as indicated at 528. In some embodiments, a rate associated with the vital sign may be calculated/estimated by calculating an average/mean, weighted mean, or median distance between adjacent Doppler spectrum peaks in each of the range bins/data collections of interest, in some cases of a predetermined period of time.

Each of the various breathing rates or other vital signs of each of the bins or other data collections may then be combined into a single, current vital sign at 532. Again, this may be accomplished by using the target range bin/data collection, or the target range bin/data collection and a certain number of adjacent or otherwise sufficiently related range bins/data collections, as previously mentioned. The current vital sign may be processed, for example, as a rolling average/mean, weighted mean, or median over a predetermined time period.

In some embodiments and implementations, the breathing rate or other vital sign may be filtered at 534 before being transmitted to another module and/or component of the vehicle, for example, as indicated in FIG. 5. This may be accomplished, for example, using a filter, such as an alpha-filter, a Kalman filter, or the like. The resulting vital sign data may then be used to take a variety of actions and/or parameter changes, as described in greater detail below.

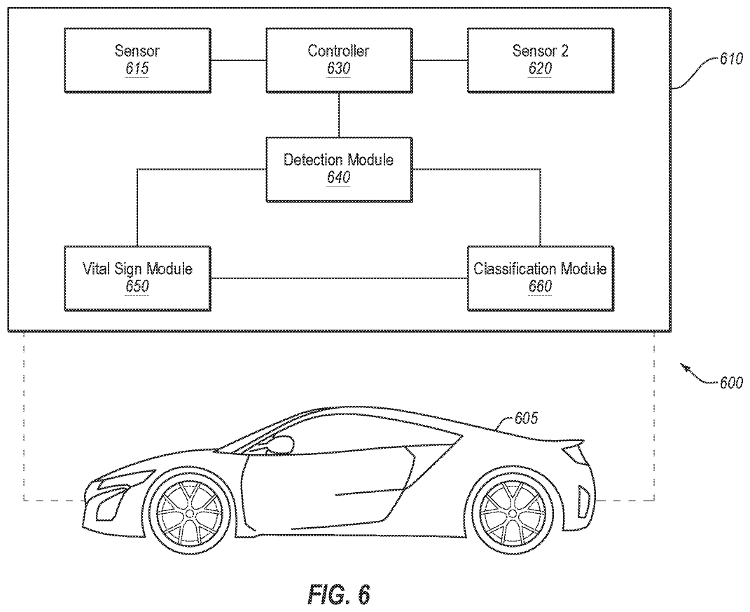

FIG. 6 illustrates an example of a system 600 for estimating a vital sign of an occupant in a vehicle 605. System 600 may comprise an internal system 610, which may combine a combination of various hardware, software, firmware, and the like as desired. System 600 comprises a first sensor module 615 and a second sensor module 620. Those of ordinary skill in the art will appreciate that, although two sensors/sensor modules are shown in the depicted embodiment, the number of sensors may vary as desired without departing from the primary inventive principles of the system 610, including a single sensor or more than two sensors.

First sensor module 615 and second sensor module 620 may comprise any number of sensors, such as RADAR sensors, LIDAR sensors, or other sensors configured to send and/or receive electromagnetic radiation, as desired. Sensor modules 615 and 620 may further comprise various other software, hardware, and/or firmware elements as desired in order to send and receive signals for processing by other modules. Although preferred embodiments may comprise and/or be limited to electromagnetic radiation sensors, it is contemplated that sensors 615, 620, and 625 may instead, in some embodiments, comprise any other sensor as desired, such as a scale/weight/pressure sensor, temperature sensor, or the like, for example.

System 610 further comprises a controller 630, which may be configured to process data from sensor modules 615/620. As used herein, the term “controller” refers to a hardware device that includes a processor and preferably also includes a memory element. The memory may be configured to store one or more of the modules referred to herein and the controller 630 and/or one or more processors may be configured to execute the modules to perform one or more processes described herein.

System 610 further comprises a detection module 640 that is coupled with both of the sensor modules 615/620. Of course, in some embodiments, a separate detection module may be provided for each sensor and/or sensor module, if desired. Detection module 640 may be configured to receive raw, sensed data from the sensors of sensor modules 615/620 and attempt to identify/detect occupants within vehicle 605 using such data, such as by detecting evidence of breathing or another vital sign, as described above and throughout this disclosure. Although in preferred embodiments system 610 may be configured specifically to detect human occupants, it is contemplated that the principles herein may also be used to detect other living occupants of a vehicle, such as dogs, cats, or other pets.

Detection module 640 may be communicatively coupled with a vital sign module 650. Vital sign module 650 may be configured to process incoming data so as to identify a vital sign, such as a breathing rate or heart rate, of an occupant and estimate the rate, as described above. Thus, in some embodiments, vital sign module 650 may be configured to perform each, or a least a subset, of the steps/processes in the system 510 of FIG. 5, as described in detail above. The resulting vital sign may then be used by system 610 to modify one or more features/parameters of vehicle 605 and/or to otherwise take actions based upon such data.

In some embodiments, this data may be used by a classification module 660 to classify the vehicle occupant associated with a particular estimated vital sign. Of course, in some embodiments, classification module 660 may be configured to classify each occupant within vehicle 605 based upon a separate vital sign associated with each vehicle occupant. As an example of a useful classification based at least partially on a breathing rate or other vital sign, classification module 660 may be configured to classify the occupant(s) as an infant, child, and/or adult. In some embodiments, for example, classification module 660 may be configured with one or more predetermined ranges of breathing rates or other breathing rates based upon statistical data correlating age with such vital sign, such as the data depicted in FIG. 4.

In some embodiments, classification module 660 may be configured to use a statistical analysis of the incoming vital sign data alone to classify the occupant(s). Alternatively, other parameters and/or features may be used in conjunction with the parameter/feature derived from the statistical analysis to classify occupants, such as data indicative of a size/weight of occupants, which could also be derived from the same RADAR sensor or other electromagnetic radiation data. Alternatively, such data may be obtained from other sensors, such as weight sensors, temperature sensors, cameras, and the like. Thus, it should be understood that the term sensor in, for example, FIG. 6, should be considered to encompass, in some contemplated embodiments, such other sensors. However, it should also be understood that, in some preferred embodiments, this term may be limited to electromagnetic sensors, such as RADAR sensors.

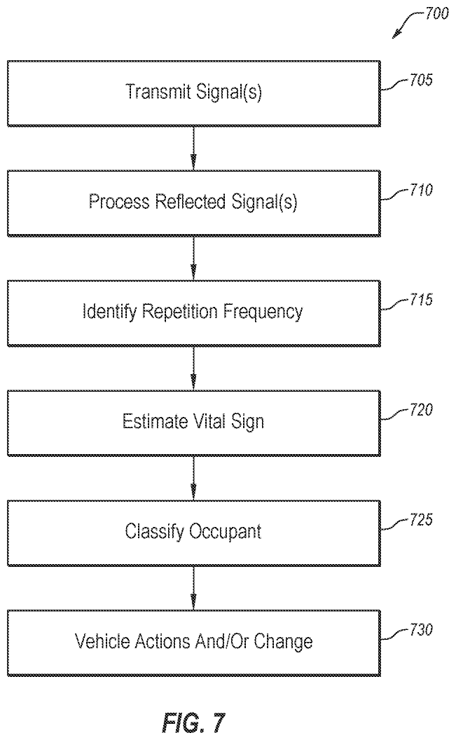

FIG. 7 is a flow chart depicting an example of a method 700 for using electromagnetic radiation to detect and estimate a vital sign of a vehicle occupant according to some implementations. Method 700 may begin with the transmission 705 of various electromagnetic signals, such as RADAR signals, from one or more sensors, as described above. These signals may, in some implementations, be directed to specific locations corresponding with seats of the vehicle, may be beamformed using multiple sensors, or may be distributed more widely throughout the vehicle so as to detect possible occupants located elsewhere in the vehicle. Also, a single sensor may be used to transmit signals to a plurality of seats/locations or multiple sensors may be used as desired. For example, a separate sensor may be used for each seat, a separate sensor may be used for each row of the vehicle, or one or more sensors may be positioned along a central portion of the vehicle from one lateral side of the vehicle to the other, as shown in FIG. 1.

Signals may then be received and/or processed at 710. In some implementations, these signals may comprise reflected signals, but this need not be the case for all contemplated implementations. Rather, in some implementations, step 710 may comprise receiving a signal at a second sensor sent from a first sensor.

A repeating frequency may then be identified at 715. As previously described, in some embodiments, this may be accomplished by identifying the peaks in a Doppler spectrum associated with one or more range bins and/or data collections. For example, RADAR data may be sorted into bins based upon, for example, range or any other suitable parameter and then the signal or signals with an identifiable repeating peak pattern that is the strongest may be identified and processed.

Such processing may be used to estimate a vital sign at 720. For example, as described above and throughout this disclosure, a vital sign may be associated with a repeating signal pattern and/or a particular occupant and then the distance between adjacent peaks in the pattern, or a statistical analysis of such pattern(s), such as interpolation, mean, weighted mean, and/or median, for example, of a distance between adjacent peaks and/or an initial vital sign rate estimate, may be used to determine and/or refine the vital sign estimate. In some implementations, additional processing steps, such as applying smoothing filters of the vital sign estimate, may also take place as part of step 720. Preferably, this vital sign estimate is then processed and refined over time to maintain a real time, or at least substantially real time estimation of the vital sign of one or more occupants in the vehicle.

Once a vital sign estimate has been obtained, which may be accomplished in step 720 or, in some implementations, based on an initial estimate of the repetition frequency of step 715, one or more occupants of the vehicle may be classified using, at least in part, the vital sign estimate, as indicated at 725 in method 700. For example, in some implementations, vehicle occupants may be classified based upon their predicted age group, which may be based wholly, or at least partially, on the vital sign estimate. This may involve use of vital sign thresholds and/or vital sign ranges. For example, if a detected breathing rate is at least 30 breaths per minute, the associated vehicle occupant may be classified as a child. In some implementations, the classification may require a stable rate detection, such as an estimation within the threshold and/or range over a predetermined period of time, so as to prevent temporary increases in breathing rate or another vital sign from re-classifying an occupant.

Occupants may also be classified based upon a threshold associated with old, rather than young, age, and/or health conditions that may be associated with a particular vital sign and/or vital sign rate. For example, if a sufficiently low, and preferably stable, breathing rate or other vital sign is detected, an occupant may be classified as a senior.

As another example of a possible classification based at least partially on a rate associated with a vital sign estimated using RADAR or another electromagnetic wave signal, in some implementations and embodiments, occupants may be classified based upon a detected change in a vital sign. For example, if a breathing rate, heart rate, or another detected vital sign drops by a predetermined amount, such a predetermined percentage or predetermined raw number of breaths/beats per minute in a relatively simple example, the occupant may be classified as sleeping or otherwise having a noteworthy condition. In some implementations and embodiments, the method/system may be configured to make this classification only for the driver, since other occupants sleeping may not be of concern. Some implementations and embodiments may also, or alternatively, be configured to detect threshold increases in vital signs over time, which may be used to classify an occupant as having a panic attack or another noteworthy condition.

Some vehicles/systems/methods may then be configured to take automated actions based upon vehicle occupant classifications and/or re-classifications at 730. For example, some vehicles may be configured to automatically disable airbags associated with a seat in which an occupant has been classified as a child/infant and/or in which no occupant vital sign can be detected.

Similarly, some embodiments and implementations may be configured to monitor the presence, or lack thereof, of an occupant and take one or more actions based at least partially thereon, which may be considered within the scope of various contemplated implementations of method 700. For example, after classifying an occupant as a child or infant, upon detecting that the vehicle has been stopped, turned off, and/or the driver and/or other occupants have exited a vehicle, in some cases after a threshold period of time, the vehicle may be configured to send a warning/notification, turn on an air conditioning unit or heater, and/or notify relevant authorities upon detecting that a child has been left in the vehicle. In some cases, this warning/notification/action may only take place upon detecting other conditions, such as a sufficiently high, or low temperature. Similarly, in some cases, the warning/notification/action may only take place after a sufficiently long period of time has elapsed since the child has been left. This time period, however, may be reduced or eliminated depending upon the estimated age of the occupant, the temperature, and/or other conditions. The time, temperature, and/or other conditions needed to trigger a warning/notification/action may scale with the projected age of the occupant. For example, the threshold time and/or temperature from room temperature needed to trigger a warning/notification/action may decrease as the projected age of the occupant decreases.

As another example, some vehicles/systems/methods may be configured to automatically take an action based upon a detected change in vital sign, such as a sufficiently dramatic increase or decrease in the vital sign. Such action(s) may comprise, for example, triggering a warning/notification/action, either within the vehicle or to a device remote from the vehicle, such as a smartphone. In some embodiments, if a dramatic increase or decrease in the vital sign, or another vital sign condition that is indicative of danger, is detected, the vehicle may be configured to take control from the driver (or, in the case of an autonomous vehicle, simply reconfigure a current driving instruction set) to slow the vehicle, pull the vehicle to the side of the road, and/or stop the vehicle.

As another example, in some embodiments and implementations, if a vital sign or vital sign change is indicative of a problematic condition, such as an elderly occupant or one or more health conditions, the vehicle may be configured to enhance monitoring of the vital sign by, for example, tuning the RADAR to more specifically and/or more closely monitor the vital sign and/or other vital signs of the vehicle occupant associated with the problematic vital sign. In some cases, the vehicle may be configured to, additionally or alternatively, target other monitoring systems, sensors, or the like for the particular occupant of concern.

For example, to assist in monitoring a possible health condition or other condition concerning for the safety of the vehicle occupants, detection of a particular vital sign or vital sign change associated with a particular occupant (again, the driver of the vehicle may warrant more attention and therefore less stringent requirements to trigger a warning/action than other occupants) may trigger actuation of another sensor and/or monitor, such as a camera, within the vehicle.

As another example, a vehicle may be configured to detect the presence of an unexpected occupant, such as a vital sign where none would be expected, for security reasons. For example, if the vehicle has been turned off, shut down, or locked without a subsequent unlocking and/or proper re-starting of the vehicle, the vehicle may be configured to monitor for vital signs and, upon detection of a vital sign under these or other circumstances under which a vital sign would not be expected, the vehicle and/or an associated application and/or system may be configured to report the incident to the owner of the vehicle and/or the police or another suitable authority to provide enhanced security.

As yet another example, some embodiments and implementations may be configured to specifically identify vital signs of living creatures other than humans, or in some cases vital signs of humans below a threshold age either along with or as an alternative to vital signs of non-humans. Upon detection of the presence of such non-human occupants—such as dogs, cats, or other pets—and/or human occupants not expected to be able to be able to open doors and/or otherwise take care of themselves, the vehicle may be configured to provide protection to such occupants. Again, this protection may be provided by providing automated temperature adjustment, which may be paired with temperature sensing to ensure that a suitable temperature in the vehicle is maintained to protect such occupants. Alternatively, or additionally, such protection may be provided by providing suitable notifications, warnings, and the like, which may be provided to an owner/operator of the vehicle, such as by way of a mobile application on a smart phone, notifications to relevant authorities, and the like.

In some embodiments and implementations, vital sign detection data may be fused with data from other types of sensors, such as cameras or any other sensors available to those of ordinary skill in the art, in order to provide further verification to authenticate the detection of a particular living occupant, human or otherwise, within the vehicle.

As used herein, a software module or component may include any type of computer instruction or computer executable code located within a memory device and/or m-readable storage medium. A software module may, for instance, comprise one or more physical or logical blocks of computer instructions, which may be organized as a routine, program, object, component, data structure, etc., that perform one or more tasks or implements particular abstract data types.

In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices. In addition, data being tied or rendered together in a database record may be resident in the same memory device, or across several memory devices, and may be linked together in fields of a record in a database across a network.

Furthermore, embodiments and implementations of the inventions disclosed herein may include various steps, which may be embodied in machine-executable instructions to be executed by a general-purpose or special-purpose computer (or another electronic device). Alternatively, the steps may be performed by hardware components that include specific logic for performing the steps, or by a combination of hardware, software, and/or firmware.

Embodiments and/or implementations may also be provided as a computer program product including a machine-readable storage medium having stored instructions thereon that may be used to program a computer (or other electronic device) to perform processes described herein. The machine-readable storage medium may include, but is not limited to: hard drives, floppy diskettes, optical disks, CD-ROMs, DVD-ROMs, ROMs, RAMS, EPROMS, EEPROMs, magnetic or optical cards, solid-state memory devices, or other types of medium/machine-readable medium suitable for storing electronic instructions. Memory and/or datastores may also be provided, which may comprise, in some cases, non-transitory machine-readable storage media containing executable program instructions configured for execution by a processor, controller/control unit, or the like.

The foregoing specification has been described with reference to various embodiments and implementations. However, one of ordinary skill in the art will appreciate that various modifications and changes can be made without departing from the scope of the present disclosure. For example, various operational steps, as well as components for carrying out operational steps, may be implemented in various ways depending upon the particular application or in consideration of any number of cost functions associated with the operation of the system. Accordingly, any one or more of the steps may be deleted, modified, or combined with other steps. Further, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced, are not to be construed as a critical, a required, or an essential feature or element.

Those having skill in the art will appreciate that many changes may be made to the details of the above-described embodiments without departing from the underlying principles of the invention. The scope of the present inventions should, therefore, be determined only by the following claims.

1. A method for detection of an occupant vital sign from within a cabin of a vehicle using RADAR, the method comprising the steps of:
identifying a repeating pattern of Doppler spectrum peaks in RADAR signal over a plurality of different range bins;
identifying an estimated frequency distance between adjacent peaks of the repeating pattern; and
calculating an estimated rate of a repeating vital sign of an occupant within the cabin of the vehicle using the estimated frequency distance.

2. The method of claim 1, wherein the estimated repeating vital sign comprises a breathing rate.

3. The method of claim 1, wherein the estimated repeating vital sign comprises a heart rate.

4. The method of claim 1, wherein the step of identifying a repeating pattern of Doppler spectrum peaks comprises selecting a strongest repeating signal from among a plurality of RADAR signals.

5. The method of claim 4, further comprising:
selecting at least one range bin adjacent to a range bin associated with the strongest signal; and
identifying an estimated frequency distance between adjacent peaks of a repeating pattern from a RADAR signal in the at least one range bin adjacent to the range bin associated with the strongest signal.

6. The method of claim 5, further comprising using the estimated frequency distance between adjacent peaks of a repeating RADAR signal in the at least one range bin adjacent to the range bin associated with the strongest signal to improve accuracy of at least one estimated parameter derived from the repeating RADAR signal.

7. The method of claim 6, wherein the at least one estimated parameter comprises a location of the occupant within the vehicle.

8. The method of claim 1, further comprising classifying the occupant using the estimated repeating vital sign.

9. A method for detection of an occupant vital sign from within a cabin of a vehicle, the method comprising the steps of:
transmitting one or more electromagnetic signals within a cabin of a vehicle;
processing signals associated with the one or more electromagnetic signals;
identifying a signal repetition frequency from the signals associated with the one or more electromagnetic signals, wherein the signal repetition frequency is indicative of a vital sign of an occupant within the cabin of the vehicle; and
estimating a vital sign of the occupant using the signal repetition frequency.

10. The method of claim 9, wherein the signals associated with the one or more electromagnetic signals comprise reflected signals, and wherein the step of processing reflected signals comprises processing reflected signals from the one or more electromagnetic signals in a plurality of range bins.

11. The method of claim 10, wherein the step of processing reflected signals comprises processing reflected signals from the one or more electromagnetic signals in a first range bin corresponding with a target range bin and a second range bin adjacent to the target range bin.

12. The method of claim 11, further comprising identifying the target range bin by comparing signal strengths of the reflected signals.

13. The method of claim 9, wherein the one or more electromagnetic signals comprise RADAR signals.

14. The method of claim 9, further comprising transmitting one or more electromagnetic signals within a cabin of a vehicle to an intended direction corresponding with an anticipated location of an occupant.

15. The method of claim 9, wherein the vital sign comprises a breathing rate.

16. A system for in-cabin detection of occupant vital signs using electromagnetic signal processing, comprising:
an electromagnetic sensor positioned within a cabin of a vehicle;
a detection module configured to process reflected electromagnetic signals into a plurality of range bins; and
a vital sign module configured to use a signal repetition frequency associated with one or more of the range bins and estimate a rate associated with a vital sign of an occupant within the cabin of the vehicle.

17. The system of claim 16, wherein the electromagnetic sensor comprises a RADAR sensor.

18. The system of claim 16, further comprising a classification module configured to receive information from the vital sign module and classify the occupant according to an age group using the rate associated with the vital sign of the occupant.

19. The system of claim 16, wherein the vital sign module is configured to estimate a breathing rate of the occupant.

20. The system of claim 16, wherein the electromagnetic sensor is configured to tune a signal to a target location expected to correspond with a location of the occupant.
